(19) United States
(12) Patent Application Publication
(10) Pub. No.: US 2022/0369936 A1
(43) Pub. Date: Nov. 24, 2022

(54) BLOOD PRESSURE MEASURING DEVICE AND METHOD

(71) Applicant: BIOBEAT TECHNOLOGIES LTD., PETACH TIKVA (IL)

(72) Inventors: ARIK BEN ISHAY, ZORAN (IL); ISRAEL SARUSSI, GANEI TAL (IL); JOHANAN MAY, PETACH TIKVA (IL)

(21) Appl. No.: 17/327,718

(22) Filed: May 23, 2021

(51) Int. Cl. A61B 5/021; A61B 5/024; A61B 5/361; A61B 5/0205; A61B 5/02; A61B 5/00; A61B 5/282

(52) U.S. Cl. CPC A61B 5/021; A61B 5/02416; A61B 5/361; A61B 5/02055; A61B 5/02042; A61B 5/6823; A61B 5/6824; A61B 5/6828; A61B 5/6833; A61B 5/282; A61B 2562/0271; A61B 2560/0223

(57) ABSTRACT

A blood pressure (BP) measuring device including a PPG sensor, having one or more light sources and one or more light detectors; a computing unit, including a receiver for receiving PPG signals from the PPG sensor and a sampling circuit, for generating PPG signals samples of the PPG signals, where the device also includes a processor having BP calculation functionality, for processing the PPG signals samples into sequential BP values and a BP output unit, for outputting the calculated BP values, where the sampling circuit is adapted to sample at high sampling rate and provide BP values at a rate higher than 1 BP value per second, where the device may also include an electrogram sensor, having one or more electrodes for outputting tissue electrical activity values, the computing unit is connected to the electrogram sensor.

### FIELD AND BACKGROUND

The invention, in some embodiments thereof, relates to blood pressure measurement and, more particularly, but not exclusively, to wearable devices for noninvasive measuring of blood pressure.

Recording sufficient BP values and related physiological data over an extended period remains a significant challenge to healthcare providers. Extended period monitoring enables a physician to identify cardiac conditions, specifically, rhythm disorders, and other physiological events of potential concern. BP monitoring tools can be improved by using long-term extended wearable BP monitoring devices.

A photoplethysmogram (PPG) is an optically obtained plethysmogram that can be used to detect blood volume changes in the microvascular bed of tissue. With the cardiac cycles the heart pumps blood to the periphery and distend the arteries and arterioles in subcutaneous tissues. The change in volume caused by the pressure pulse can be detected by illuminating the skin with a light source (LED) and then measuring the amount of light either transmitted or reflected to a photodiode.

It has been a problem to maintain continual contact between PPG or electrogram electrodes and the skin after a day or two. Dirt, moisture, and other environmental contaminants, as well as perspiration, skin oil, and dead skin cells from the patient's body, can get between the electrode and the skin's surface. These factors may reduce the quality of signal recordings. Physical movements of the patient and their clothing impart various compressional, tensile, and torsional forces on the contact point of the electrodes, especially over long recording times, and an inflexibly fastened electrodes will be prone to becoming dislodged. Dislodgment may occur unbeknownst to the patient, making the BP or electrogram recordings worthless Thus, it is desired to periodically remove or replace the electrodes during a long-term BP or electrogram monitoring period.

### SUMMARY

In an embodiment of the invention a blood pressure (BP) measuring device is provided, including a PPG sensor, having one or more light sources and one or more light detectors, a computing unit, connected to the PPG sensor, including a receiver for receiving PPG signals from the PPG sensor, a sampling circuit, for generating PPG signals samples of the PPG signals, a processor having BP calculation functionality, for processing the PPG signals samples into sequential BP values, a BP output unit, for outputting the calculated BP values, wherein the sampling circuit is adapted to sample at high sampling rate and provide BP values at a rate higher than 1 BP value per second.

In some cases, the device further including an electrogram sensor, having one or more electrodes for outputting tissue electrical activity values, wherein the computing unit is connected to the electrogram sensor.

In some cases, the BP output unit is adapted to output sequential BP values for at least 1 minute and/or the sampling circuit is adapted to sample at least 100 consecutive PPG signals. In some cases, the computing unit includes a storage unit for storing the PPG signals received within a time range of at least 1 minute, and the BP calculation functionality includes processing the PPG signals stored in the storage unit.

In some cases, the BP output unit provides BP values only when systolic blood pressure is higher than set threshold. In some cases, the BP output unit provides BP values only when diastolic blood pressure is lower than set threshold. In some cases, the BP output unit provides BP values only when the heart rate is greater than a threshold or below a threshold. In some cases, the BP output unit provides BP values only when detecting cardiac arrhythmia or detecting bleeding. In some cases, the BP output unit provides BP values only during normal sinus rhythm. In some cases, the BP output unit provides BP values only during atrial fibrillation. In some cases, the BP output unit provides BP values only during non-epileptic periods or during epileptic seizure. In some cases, the BP device includes a temperature sensor, and the processed. BP output unit provides BP values only for selected ranges of body temperature values measured by the temperature sensor.

In some cases, the processed BP output unit provides BP values only when detecting cardiac arrhythmia or detecting bleeding. In some cases, the BP output unit provides BP values only when HR is with one or more selected HR ranges. In some cases, the BP output unit provides BP values only when breathing rate within a set one or more breathing rate range. In some cases, the BP output unit provides BP values only when BP variability (BPV) is within a set value range and/or above and/or below a set threshold.

In some cases, the BP device measures SV and or CO, and the BP output unit provides BP values only when SV and/or CO is within a set value range and/or above and/or below a set threshold. In some cases, the electrogram sensor includes two or more electrodes, and the two or more electrodes are located on 2 sides of the PPG sensors, in a distance less than 100 mm from the PPG sensor. In some cases, the BP output unit is activated when the tissue electrical activity values are within a pre-defined range.

In some cases, the device is a wearable device. In some cases, the device is wearable on a limb or a chest. In some cases, the device is attachable to a tissue or body organ. In some cases, the device includes an adhesive layer, said adhesive layer is attachable to the skin.

In some cases, the device further including a communication circuit adapted to communicate with one or more other BP devices, and having a receiver circuit for receiving data from the one or more other BP devices.

In some cases, the device further including calibration functionality adapted to receive data from the one or more other BP devices, and to output calibration data. In some cases, the calibration functionality receives as an input calibration data of the one or more other BP devices, and processes calibration data. It is another object of the subject matter to disclose a blood pressure (BP) measuring system, including a first and a second blood BP measuring devices of any one of the above examples, operating simultaneously.

It is another object of the subject matter to disclose a method for measuring blood pressure using a BP measuring device having a PPG sensor and a computing unit, the method including receiving PPG signals from the PPG sensors, sampling by the computing unit the received PPG signals into PPG signals samples at a rate higher than 120 Hz, processing the PPG signals samples into BP values by the computing unit, repeating the sampling for at least 100 cycles, and outputting the BP values.

In some cases, the method further including measuring a tissue electrical activity by an electrogram sensor included with the BP measuring device, and wherein the outputting is activated when the electrical activity is within a pre-defined range.

In some cases, the method further including receiving calibration data by a receiver circuit provided in the BP measuring device, and calibrating the BP measuring device to an operational profile that matches the calibration data.

In some cases, the receiving is from PPG sensors coupled to a body tissue. In some cases, the receiving is performed from PPG sensors assembled within a wearable BP measuring device In some cases, the receiving is performed from PPG sensors assembled within a body patch BP measuring device and attached to the body tissue. In some cases, the measuring of blood pressure is performed by using two or more of the BP measuring devices, and including operating simultaneously the two or more of the BP measuring devices, and communicating signals data between the two or more of the BP measuring devices.

In some cases, the method further including coupling a first of the two or more BP measuring device on a limb, and coupling a second of the two or more BP measuring device on a body tissue. In some cases, the method further including coupling the two or more BP measuring device on one or more limbs.

In some cases, the receiving is performed when systolic pressure is above 140 mmHg. In some cases, the receiving is performed when diastolic pressure is above 90 mmHg. In some cases, the receiving is performed when systolic pressure is below 100 mmHg. In some cases, the receiving is performed when diastolic pressure is below 80 mmHg. In some cases, the receiving is performed when HR and cardiac arrhythmia is detected using the PPG sensor only.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of a blood pressure measuring device, according to exemplary embodiments of the subject matter;

FIG. 2A which illustrates a bottom view of a wearable apparatus having a BP device illustrated in isometric view in FIG. 2B, according to exemplary embodiments of the subject matter;

FIGS. 3A and 3B which illustrate isometric views of a BP device in a band wearable form, according to exemplary embodiments of the subject matter;

FIGS. 4A and 4B which are diagrams of some examples of processes for measuring BP, according to some embodiments of the subject matter.

### DETAILED DESCRIPTION

The invention, in some embodiments thereof, relates to blood pressure measurement and, more particularly, but not exclusively, to wearable devices for noninvasive measuring of blood pressure.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

In one embodiment of the invention, there is provided a blood pressure measuring device.

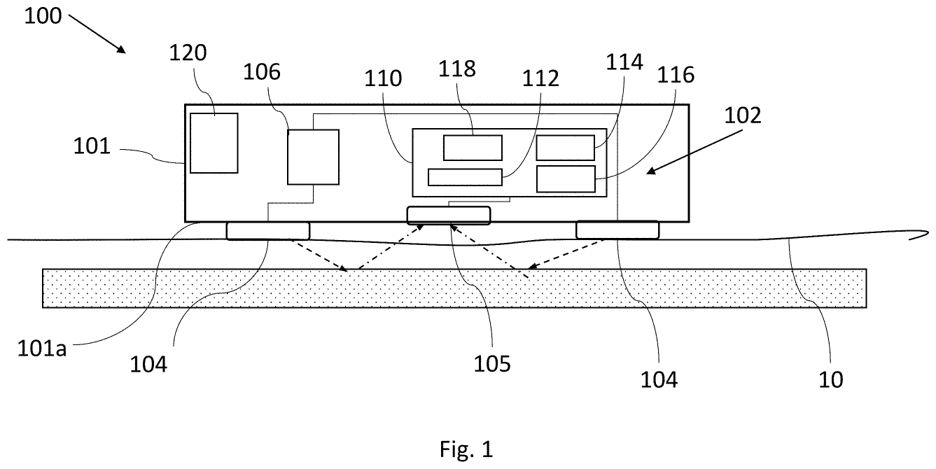

Referring now to the drawings, FIG. 1 is a schematic illustration of a blood pressure measuring device, according to exemplary embodiments of the subject matter.

In one embodiment of the invention, there is provided a blood pressure measuring device (referred to hereinafter as BP device). As shown in FIG. 1, blood pressure measuring device 100 comprises a housing 101, and a PPG sensor 102 coupled to housing 101. PPG sensor 102 has one or more light sources 104 and one or more light detectors 105.

According to some embodiments, light sources 104 and/or light detectors 105 are exposed to tissue 10. In some embodiments, as shown in FIG. 1, light sources 104 and/or light detectors 105 are exposed to tissue 10 via a tissue facing surface 101a of housing 101.

According to some embodiments, BP device 100 has a computing unit 110, connected to PPG sensor 102 (e.g. to detectors 105) and comprises a PPG receiver 112 for receiving PPG signals from PPG sensor 102. Computing unit 110 comprises a sampling circuit 114, for generating PPG signals samples of the received PPG signals. Computing unit 110 comprises a processor 116 having a BP calculation functionality, for processing the PPG signals samples into a BP profile. The BP profile may include BP parameters such as sequential BP values, BP change rate, diastolic pressure values, systolic pressure values, maximum BP, and minimum BP.

According to some embodiments, PPG sensor 102 has a PPG actuation circuit 106 to control the light source 104 actuation signals. In some embodiments, PPG actuation circuit 106 is activated by computing unit enclosed within BP device 100, In some embodiments, PPG actuation circuit 106 is activated by a source outside BP device 100.

According to some embodiments, computing unit 110 further comprises a BP output unit 118, for outputting the calculated values of the BP profile.

According to some embodiments, computing unit 110 further comprises a storage unit for storing the PPG signals. In some embodiments, storage unit is adapted to store PPG signals and/or BP values received within a time range of at least 1 minute. In some embodiments, storage unit is adapted to store PPG signals received within a time range of at least 2 minutes. In some embodiments, storage unit is adapted to store PPG signals received within a time range of at least 10 minutes. In some embodiments, the BP calculation functionality includes reading and processing the PPG signals stored in the storage unit.

According to some embodiments, sampling circuit 114 is adapted to sample at high sampling rate and provide BP values at a rate higher than 1 BP value per second. In some embodiments, sampling circuit 114 samples the PPG signals at a sample rate higher than 120 Hz. In some embodiments, sampling circuit 114 samples the PPG signals at a sample rate higher than 0.0001 Hz. In some embodiments, sampling circuit 114 samples the PPG signals at a sample rate higher than 0.001 Hz. In sonic embodiments, sampling circuit 114 samples the PPG signals at a sample rate higher than 0.01 Hz. In some embodiments, sampling circuit 114 samples the PPG signals at a sample rate higher than 0.1 Hz. In some embodiments, sampling circuit 114 samples the PPG signals at a sample rate higher than 1 Hz. In some embodiments, sampling circuit 114 samples the PPG signals at a sample rate higher than 10 Hz. In some embodiments, sampling circuit 114 is adapted to sample at least 10 consecutive PPG signals. In some embodiments, sampling circuit 114 is adapted to sample at least 100 consecutive PPG signals. In some embodiments, sampling circuit 114 is adapted to sample at least 1000 consecutive PPG signals. Such sampling range may allow continuous BP measuring and monitoring.

In some embodiments, BP output unit 118 is adapted to output BP profile values of at least 1 minute of sequential BP measuring. In some embodiments, BP output unit 118 is adapted to output BP profile values of least 5 minutes of sequential BP measuring, In some embodiments, BP output unit 118 is adapted to output BP profile values of at least 10 minutes of sequential BP measuring. Such output cycle may allow continuous BP measuring and monitoring.

The BP device, according to an embodiment of the invention can be used for continues BP monitoring for a long period of time. The example operational parameters provided elsewhere herein are for demonstrating the continuous BP monitoring.

According to some embodiments, output unit 110 provides BP values only when systolic blood pressure is higher than a set threshold. In some embodiments, the BP profile values include systolic blood pressure higher than 140 to 160 mmHg. According to some embodiments, output unit 110 provides BP values only when diastolic blood pressure is higher than a set threshold. In some embodiments, the BP profile values include BP values include diastolic blood pressure higher than 90 to 110 mmHg. According to some embodiments, output unit 110 provides BP values only when diastolic blood pressure is lower than a set threshold. According to some embodiments, output unit 110 provides BP values only when systolic blood pressure is lower than a set threshold. In some embodiments, the BP profile values include systolic blood pressure lower than 80 to 100 mmHg, According to some embodiments, output unit 110 provides BP values only when diastolic blood pressure is lower than a set threshold. In some embodiments, the BP profile values include diastolic blood pressure lower than 60 to 80 mmHg.

According to some embodiments, output unit 110 provides BP values only when one or more of the following is detected: heart rate is greater than a threshold, heart rate is below a threshold, cardiac arrhythmia, bleeding. In some embodiments, output unit 110 provides BP values only when one or more of the following parameters are with a programmable range: BP, HR, respiratory rate, HRV, pulse pressure, mean arterial pressure, stroke volume, cardiac output, cardiac index, systemic vascular resistance, body temperature, blood oxygen saturation.

According to some embodiments, BP device 100 further comprises a communication circuit 120 adapted to communicate with one or more other BP devices (e.g. like BP device 100, or others connectable devices). In some embodiments, communication circuit 120 has a receiver circuit for receiving data from the one or more other BP devices. In some embodiments, communication circuit 120 has a transmitter for transmitting data to the one or more other BP devices. According to some embodiments, computing unit 110 has a calibration functionality adapted to receive data from the one or more other BP devices, and to output calibration data. In some embodiments, the calibration functionality receives as an input calibration data of the one or more other BP devices and processes the calibration data. In some embodiments, the calibration functionality receives as an input calibration data, such as systolic and diastolic values using other measuring modalities and calibrate the BP device accordingly.

According to some embodiments, BP device 100 is a wearable device as will be exemplified in the next figures. In some embodiments, BP device 100 comprises an attachment-case for coupling BP device 100 to tissue 10 (e.g. skin). In some embodiments, housing 101 is coupled with attachment-case. In some embodiments, attachment-case is attachable to tissue 10. In some embodiments, attachment-case is attachable to tissue 10 using an adhesive layer. In some embodiments, attachment-case is adapted to affix BP device 100 to tissue 10. In some embodiments, attachment-case is adapted to affix BP device 100 to tissue 10 by pressing tissue layer 101a to tissue 10. In some embodiments, BP device 100 is in the form of a bracelet. In some embodiments, BP device 100 is in the form of a watch like device.

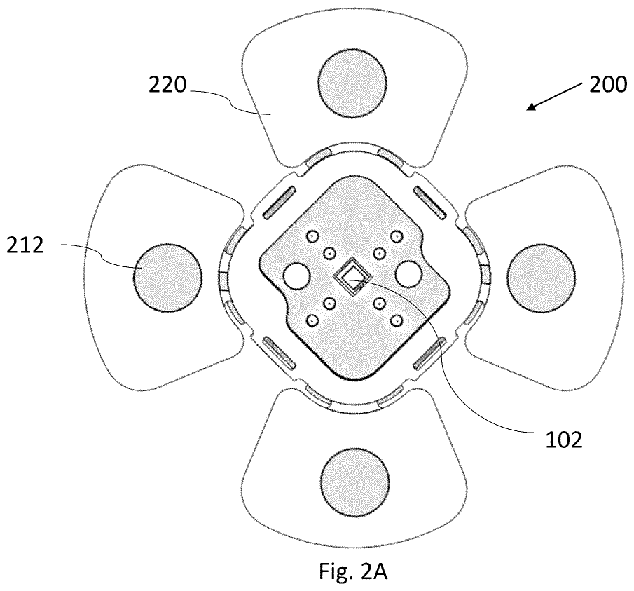

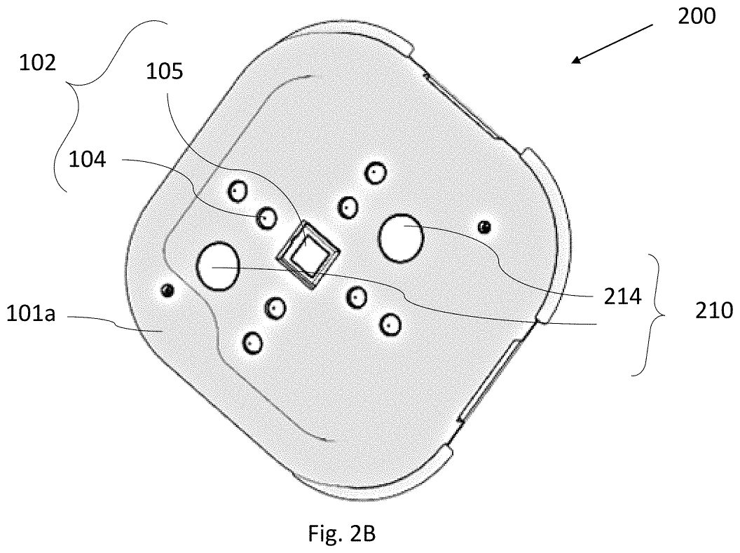

Referring now to FIG. 2A which illustrates a bottom view of a wearable apparatus having a BP device illustrated in isometric view in FIG. 2B, according to exemplary embodiments of the subject matter.

As shown in FIGS. 2A and 2B, BP device 200 has the components disclosed in BP device 100. BP device 200 further comprises an electrogram sensor, having one or more electrodes for outputting tissue electrical activity values. The electrogram sensor maybe an electrogram sensor such as an ECG, EEG, EMG, EOG, EGG. Electrical activity can be for example heart rate, EEG/EMG amplitude, impedance.

In some embodiments, as shown in FIG. 2A, electrogram sensor has two or more tissue impedance electrodes 212 placed around PPG sensor 102. In some embodiments, impedance electrodes 212 are of ECG. In some embodiments, as shown in FIG. 2B, electrogram sensor 210 has two or more tissue impedance electrodes 214 placed around light detectors 105. In some embodiments, tissue impedance electrodes 214 are used for measuring galvanic skin response (GSR).

According to some embodiments, computing unit 110 is connected to electrogram sensor 210. In some embodiments, computing unit 110 includes electrical activity functionality for calculating tissue electrical activity values in parallel to calculating the BP values. In some embodiments, connection between computing unit 110 and electrogram sensor 210 is wired. In some embodiments, connection between computing unit 110 and electrogram sensor 210 is wireless. In some embodiments, computing unit 110 further comprises a storage unit for storing the electrogram signals.

According to some embodiments, BP output unit 118 is calibrated to be activated when the electrical activity values are within a pre-defined range.

As shown in FIG. 2A BP device 200 is adapted to be a wearable device and by having a securing module 220. Attachment-case 20 is adapted to expose light sources 104 and light detectors 105 to tissue 10. Securing module 220 is configured to position BP device 200 abutting tissue 10 for a short time, for example up to 5 minutes, or may be positioned and attached to tissue 10 for a longer term (e.g. more than 10 minutes) for a continuous monitoring of the BP profile. The BP device 200 may be coupled to the tissue 10 for a few seconds, until a verified BP value is obtained. Verification can be defined, for example, by having a stable median value of the systolic or diastolic BP. Stability can be defined as a series of values changing by less than a threshold, such as 2,1, 0.5, 0.1%. In some cases, median is calculated only after having at least a predefined number of BP measurements. For example, 10, 20, 50, 100 measurements.

According to some embodiments, securing module 220 is used for housing one or more sensors and/or electrodes of such sensors. In some embodiments, as shown in FIG. 2A, an electrogram sensor has two or more tissue impedance electrodes 212 coupled to securing module 220.

According to some embodiments, BP device 200 provides indications about the BP profile and/or the operational state of the BP device by one or more of visual signals, voice signals, vibrations. In some embodiments (not shown), BP device 200 further comprises a display unit which can show values related to the measured BP profile, e.g. values provided by output unit 118.

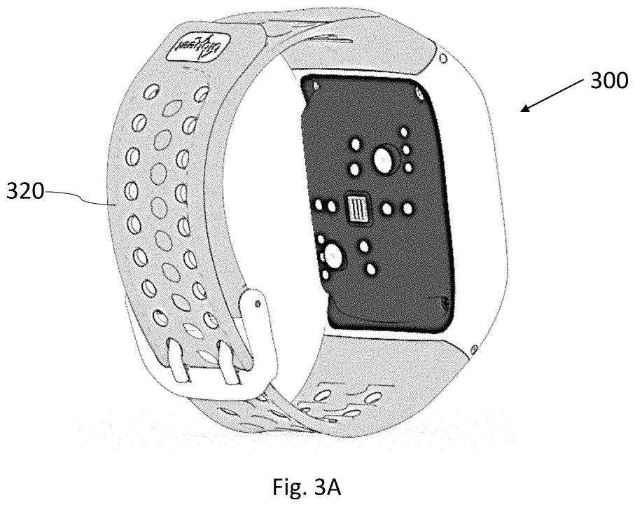

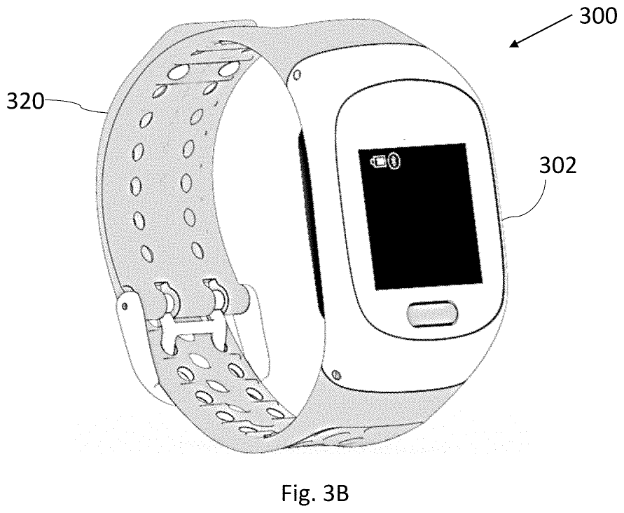

Referring now to FIGS. 3A and 3B which illustrate isometric views of a BP device in a band wearable form, according to exemplary embodiments of the subject matter.

As shown in FIGS. 3A and 3B, BP device 300 has a securing module 320 in the form of a watch. BP device 300 is structurally similar to the embodiment disclosed in BP device 200.

According to some embodiments, BP device 200 and/or 300 provides indications about the BP profile and/or the operational state of BP device 200/300 by one or more of visual signals, voice signals, vibrations and the like. In some embodiments for example as shown in FIG. 3B, BP device 200 and/or 300 further comprises a display unit which can show values related to the measured BP profile, e.g., profile values provided by output unit 118. In some embodiments, securing module 220 and/or 320 further comprises the display unit.

According to some embodiments BP device 100/200/300 includes one or more control units to control the operation of BP device 100 (e.g., PPG sensor 102, electrogram sensor 210, computing unit 110, sampling circuit 114, and output unit 118). In some embodiments, control units are configured to trigger BP measuring during specific conditions evaluated based on signals received by electrogram sensor 210. Some examples of such conditions are:

Normal cardiac sinus rate detected by electrogram sensor 210 (e.g., ECG);

Cardiac arrhythmia where the cardiac arrhythmia is detected by electrogram sensor 210 (e.g., ECG);

Epileptic condition where the epileptic seizure is detected by electrogram sensor 210 (e.g., EEG);

Sleep stages like REM, non-REM, deep sleep, detected by electrogram sensor 210 (e.g., EOG or EMG);

Sweating periods as detected by electrogram sensor 210 that monitors the change in the electrical activity of the tissue.

HR below or over a set threshold, HRV below or above a set threshold (e.g. lower than 40-60) BPV above or below a set threshold (e.g. higher than 80-140). In some embodiments, BP device 100/200/300 includes a setting functionality for setting one or more of the above thresholds.

According to some embodiments BP device 100/200/300 includes a temperature sensor and calculates BP only for selected ranges of body temperature. In some embodiments, for each range the BP value is measured separately.

In one embodiment of the invention, there is a blood pressure measuring system (BP system), comprising a first and a second BP devices as disclosed elsewhere herein.

In some embodiments, the first and second. BP devices are configured to operate simultaneously. Some examples of simultaneous operation are exchanging of calibration data, activation of one BP device when the other is not activated, measuring different ranges of BP profile value, etc.

According to some embodiments, the one or more BP devices disclosed elsewhere herein (e.g., 100-300) measure the following parameters simultaneously: HR, HRV (Heart rate variability), BP, BPV (blood pressure variability), SV, CO, CI, SVR, RR, SAT %, skin, ambient and body temperature.

In one embodiment of the invention there is a method for measuring BP using one or more BP devices disclosed elsewhere herein.

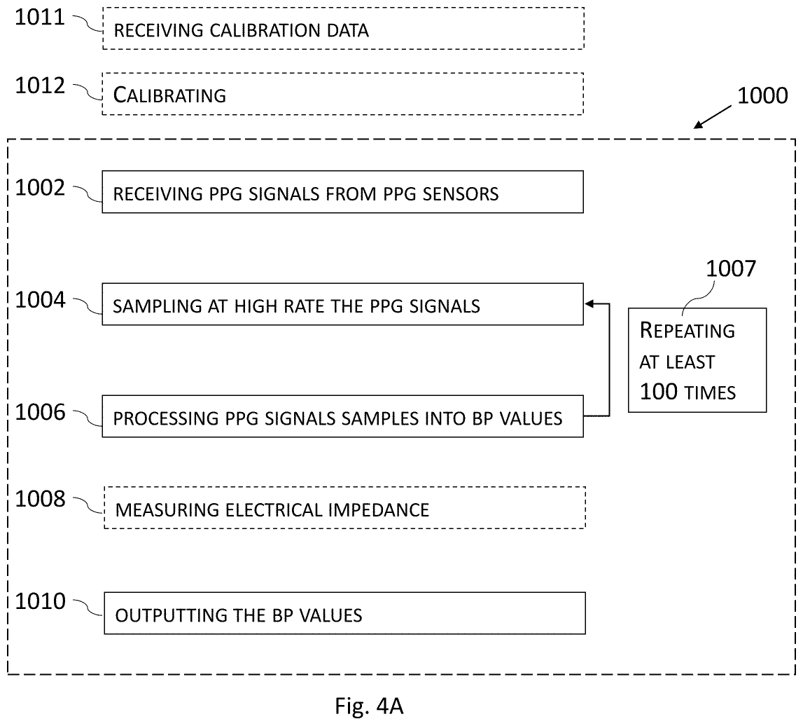

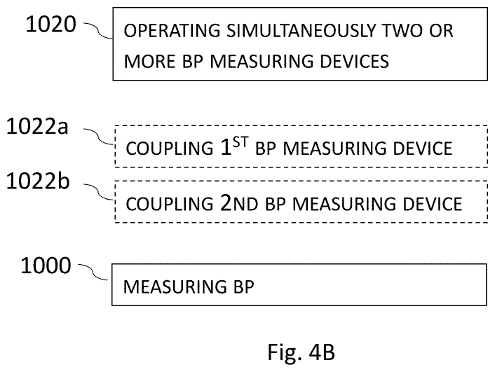

Reference is now made to FIGS. 4A and 4B which are diagrams of some examples of processes for measuring BP, according to some embodiment of the current invention. As shown in FIG. 4A, the process of BP measuring 1000 comprises the steps of:

Step 1002 discloses receiving of PPG signals from the PPG sensors of the BP device. In some embodiments, receiving the PPG signals may be performed when systolic pressure is above 140 to 160 mmHg. In some embodiments, receiving the PPG signals may be performed when diastolic pressure is above 90 to 110 mmHg. In some embodiments, receiving the PPG signals may be performed when systolic pressure is below 80 to 100 mmHg. In some embodiments, receiving the PPG signals may be performed when diastolic pressure is below 60 to 80 mmHg, in some embodiments, receiving 1002 is performed when cardiac arrhythmia is detected by the PPG sensor of the BP device.

Step 1002 discloses sampling the received PPG signals into PPG signals. In some embodiments, the process of sampling 1002 is at a high sampling rate and includes providing BP values at a rate higher than 1 BP value per second. In some embodiments, the process of sampling 1002 is performed at a rate higher than 120 Hz. Sampling may be performed by the computing unit of the BP device.

In some embodiments, the process of BP measuring 1000 further comprises processing 1006 the PPG signals samples into BP values by the computing unit and repeating 1007 the sampling process 1004. The sampling rate may change over time, or be constant. The sampling rate may be, for example, in the range of 1-10,000 samples per second. In some embodiments, repeating 1007 is performed prior to processing 1006.

In some embodiments, BP measuring 1000 further comprises outputting 1010 the BP values. In some embodiments, repeating 1007 is performed after outputting 1010.

According to some embodiments, the BP measuring 1000 optionally includes measuring 1008 the electrical activity of a tissue by an electrogram sensor included with the BP device. In some embodiments, outputting 1010 is activated when the electrical activity is within a pre-defined range. In some embodiments, electrical activity measuring 1008 is performed before or in parallel to any one of steps 1002-1007.

According to some embodiments, BP measuring 1000 further includes calibrating 1012 the BP device. Calibrating 1012 may be performed before, in parallel, or after any one of BP measuring 1000 steps 1002-1010.

According to some embodiments, calibrating 1012 follows a procedure of receiving 1011 calibration data by a receiver circuit provided in the BP measuring device. In some embodiments, receiving 1012 is performed from PPG sensors coupled to a body tissue. In some embodiments, the calibration data are received from PPG sensors assembled within a wearable BP measuring device. In some embodiments, the calibration data are received from PPG sensors assembled within a body patch BP measuring device and attached to the body tissue. Anyone of the PPG sensors, body patch BP measuring device, and the wearable BP measuring device, may be an embodiment of the BP device disclosed elsewhere herein. In some embodiments, calibrating 1012 is to an operational profile that matches the calibration data.

According to some embodiments, as shown for example in FIG. 4B, BP measuring 1000 is performed by using two or more of the BP measuring devices and comprises operating 1020 simultaneously two or more of the BP measuring devices. In some embodiments, operating 1020 is preceded by coupling 1022a a first of the two or more BP measuring device to a body tissue, such as a limb (e.g., right/left limb, ankle and wrist) or chest, and coupling 1022b a second of the two or more BP measuring device on a body tissue, a limb (e.g., right/left limb, ankle and wrist), or chest. In some embodiments, BP measuring 1000 is performed by two or more of the BP measuring devices and comprises communicating 1024 signals data between the two or more of the BP measuring devices.

According to some embodiments, continuous BP measuring 1000 is performed by one of more modalities BP measuring device in the form of a body patch, in the form of a limb bracelet, earbuds, wearable cloth like shirt, socks, shoes, hat, glasses and the like. In some embodiments during BP measuring 1000, the BP measurement device can be replaced from being one BP measurement device to another BP measurement device of the same or different type or form. In some embodiments, calibration data of one BP measurement device can be used as calibration data for another BP measurement device. According to some embodiments, the BP measured on a single person, using 2 or more devices simultaneously, may be used to improve the accuracy of the measured BP.

According to some embodiments, measuring 1000 is performed during specific medical conditions, such as: Hypertension, hypotension during dialysis, cardiac arrhythmia, sepsis, shock or trauma, and bleeding. In some embodiments, measuring 1000 is continuous during such medical conditions.

According to some embodiments measuring 1000 is initiated when one or more of the following measured parameters are within a pre-defined range: BP, HR, respiratory rate, HRV, pulse pressure, mean arterial pressure, stroke volume, cardiac output, cardiac index, systemic vascular resistance, body temperature, blood oxygen saturation. According to some embodiments. BP measuring 1000 is simultaneous with measuring other parameters such as HR, HRV (Heart rate variability), BPV (blood pressure variability), SV, CO, CI, SVR, SAT %, skin and body temperature. In some embodiments, measuring other parameters is by the BP device. In some embodiments, measuring other parameters is by another BP device.

The terms “comprises”, “comprising”, “includes”, “including”, “has”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure. As used herein, the singular forms “a”, “an” and “the” include plural references unless the context clearly dictates otherwise.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range. Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween. Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments unless the embodiment is inoperative without those elements. Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

What is claimed is:

1. A blood pressure (BP) measuring device, comprising:
a PPG sensor, having one or more light sources and one or more light detectors;
a computing unit, connected to the PPG sensor, comprising:
a receiver for receiving PPG signals from the PPG sensor;
a sampling circuit, for generating PPG signals samples of the PPG signals;
a processor having BP calculation functionality, for processing the PPG signals sampled into sequential BP values;
wherein the sampling circuit is adapted to sample at high sampling rate and provide BP values at a rate higher than 1 BP value per second.

2. The device of claim 1, further comprising:
an electrogram sensor, having one or more electrodes for outputting tissue electrical activity values;
wherein the computing unit is connected to the electrogram sensor.

3. The device of claim 1, further comprising a BP output unit, for outputting the calculated BP values.

4. The device of claim 3, wherein the BP output unit is adapted to output sequential BP values for at least 1 minute and/or the sampling circuit is adapted to sample at least 100 consecutive PPG signals.

5. The device of claim 1, wherein the computing unit comprises a storage unit for storing the PPG signals received within a time range of at least 1 minute; and the BP calculation functionality includes processing the PPG signals stored in the storage unit.

6. The device of claim 3, wherein the BP output unit provides BP values only when systolic blood pressure is higher than a set threshold or lower than a set threshold.

7. The device of claim 3, wherein the BP output unit provides BP values only when diastolic blood pressure is higher than a set threshold or lower than a set threshold.

8. The device of claims 3, wherein the BP output unit provides BP values only when the heart rate is greater than a set threshold or below a set threshold.

9. The device of claim 3, wherein the BP output unit provides BP values only when detecting cardiac arrhythmia or detecting bleeding.

10. The device of claim 3, wherein the BP output unit provides BP values only during normal sinus rhythm.

11. The device of claim 3, wherein the BP output unit provides BP values only during atrial fibrillation.

12. The device of claim 1, wherein the BP device comprises a temperature sensor and the processed BP output unit provides BP values only for selected ranges of skin or body temperature values measured by the temperature sensor.

13. The device of claims 3, wherein the processed BP output unit provides BP values only when detecting cardiac arrhythmia or detecting bleeding.

14. The device of claim 3, wherein the BP output unit provides BP values only when HR is with one or more selected HR ranges.

15. The device of claim 3, wherein the BP output unit provides BP values only when breathing rate is within a set of one or more breathing rate range.

16. The device of claim 3, wherein the BP output unit provides BP values only when BP variability (BPV) is within a set value range and/or above and/or below a set threshold.

17. The device of claim 3, wherein the BP device measures SV and or CO, and the BP output unit provides BP values only when SV and/or CO is within a set value range and/or above and/or below a set threshold.

18. The device of claim 2, wherein the electrogram sensor comprises two or more electrodes, and the two or more electrodes are located on 2 sides of the PPG sensors, in a distance less than 100 mm from the PPG sensor.

19. The device of claim 3, further comprising an electrogram sensor, having one or more electrodes for outputting tissue electrical activity values; wherein the computing unit is connected to the electrogram sensor wherein the BP output unit is activated when the tissue electrical activity values are within a pre-defined range.

20. The device of claim 1, wherein the device is a wearable device.

21. The device of claim 1, wherein the device is wearable on a limb or a chest.

22. The device of claim 1, wherein the device is attachable to a tissue or body organ.

23. The device of claim 1, wherein the device comprises an adhesive layer, said adhesive layer is attachable to the skin.

24. The device of claim 1, further comprising a communication circuit adapted to communicate with one or more other BP devices, and having a receiver circuit for receiving data from the one or more other BP devices.

25. The device of claim 24, further comprising calibration functionality adapted to receive data from the one or more other BP devices, and to output calibration data.

26. The device of claims 24, wherein the calibration functionality receives as an input calibration data of the one or more other BP devices of claim 24, and processes calibration data.

27. A blood pressure (BP) measuring system, comprising a first and a second blood BP measuring devices of claim 24, operating simultaneously.

28. A method for measuring blood pressure using a BP measuring device having a PPG sensor and a computing unit, the method comprising:
receiving PPG signals from the PPG sensors;
sampling by the computing unit the received PPG signals into PPG signals samples at a rate higher than 120 Hz;
processing the PPG signals samples into BP values by the computing unit;
repeating the sampling for at least 100 cycles; and
outputting the BP values.

29. The method according to claim 28, further comprising:
measuring a tissue electrical activity by an electrogram sensor included with the BP measuring device; and
wherein the outputting is activated when the electrical activity is within a pre-defined range.

30. The method according to claims 28, further comprising:
receiving calibration data by a receiver circuit provided in the BP measuring device, and
calibrating the BP measuring device to an operational profile that matches the calibration data.

31. The method according to claim 28, wherein the receiving is from PPG sensors coupled to a body tissue.

32. The method according to claim 28, wherein the receiving is from PPG sensors assembled within a wearable BP measuring device.

33. The method according to claims 28, wherein the receiving is from PPG sensors assembled within a body patch BP measuring device and attached to the body tissue.

34. The method according to claim 28, wherein the measuring of blood pressure is performed by using two or more of the BP measuring devices, and comprising:
operating simultaneously the two or more of the BP measuring devices, and
communicating signals data between the two or more of the BP measuring devices.

35. The method according to claim 34, further comprising:
coupling a first of the two or more BP measuring devices on a limb, and coupling a second of the two or more BP measuring device on a body tissue.

36. The method according to claim 34, further comprising:
coupling the two or more BP measuring device on one or more limbs.

37. The method according to claim 28, wherein the receiving is performed when HR and cardiac arrhythmia is detected using the PPG sensor only.

38. A blood pressure (BP) measuring device, comprising:
a PPG sensor, having one or more light sources and one or more light detectors;
a computing unit, connected to the PPG sensor, comprising:
a receiver for receiving PPG signals from the PPG sensor;
a sampling circuit, for generating PPG signals samples of the PPG signals;
a processor having BP calculation functionality, for processing the PPG signals sampled into sequential BP values;
an electrogram sensor, having one or more electrodes for outputting tissue electrical activity values;
wherein the computing unit is connected to the electrogram sensor;
wherein the device is wearable on a patient's chest.
